(19) United States
(12) Patent Application Publication
(10) Pub. No.: US 2026/0306211 A1
(43) Pub. Date: Oct. 8, 2026

(54) MAGNETIC RADIOGRAPHIC MARKER HOLDER

(71) Applicant: Mayo Foundation for Medical Education and Research, Rochester, MN (US)

(72) Inventors: Joshua E. Sellwood, Jacksonville, FL (US); David J. DiSantis, Jacksonville, FL (US); Samir A. Dabit, Jacksonville, FL (US); James M. Jeter, Jacksonville, FL (US); Catherine A. Boniface, Jacksonville, FL (US)

(21) Appl. No.: 19/638,500

(22) Filed: Apr. 3, 2026

Related U.S. Application Data
(60) Provisional application No. 63/783,422, filed on Apr. 4, 2025.

(51) Int. Cl. A61B 90/00; A61B 6/50

(52) U.S. Cl. CPC A61B 90/39; A61B 6/505; A61B 2090/3966; A61B 2090/3991

(57) ABSTRACT

Magnetic radiographic marker holders for use with x-ray imaging allow for repeatable positioning of radiographic markers relative to the patient’s anatomy, such as a cervical spinal region containing the first and second cervical vertebrae. The radiographic marker holder may include an earloop extending from a proximal end to a distal end, a marker holder body coupled to the earloop at the distal end of the earloop, and a magnet coupled to the marker holder body to receive and magnetically couple a radiographic marker to the marker holder body.

### CROSS-REFERENCE TO RELATED APPLICATIONS

This application claims the benefit of U.S. Provisional Patent Application Serial No. 63/783,422, filed on Apr. 4, 2025, and entitled “Magnetic Radiographic Marker Holder,” which is herein incorporated by reference in its entirety.

### BACKGROUND

Radiographic markers are small objects (e.g., tags) used in x-ray imaging to provide information about an x-ray image and its content. For example, radiographic markers identify details such as the side of the body being imaged, the orientation of the body part, or any other information that may assist with interpreting the x-ray image.

In the context of modified barium swallow (MBS) studies, radiopaque tags are typically placed on static surfaces to mark images and identify different consistencies being swallowed. The process of placing and changing these radiographic markers can be slow. Additionally, patient movement before, during, and after swallowing can remove the radiographic marker from view or cause it to be missed during imaging completely. The process of repeatedly placing and adjusting these radiographic markers can therefore extend the duration of the study, which can be problematic when multiple studies need to be performed in a limited time block. Additionally, the radiologist must divide their attention between finding the radiographic marker and identifying pathology in real-time.

There remains a need for the ability to consistently place radiographic markers relative to specific anatomical landmarks, and such that the placement of the radiographic markers allows for tighter collimation of the radiation beam, which can reduce radiation exposure to the patient.

### SUMMARY OF THE DISCLOSURE

According to an aspect of the present disclosure, a magnetic radiographic marker holder is provided. The magnetic radiographic marker holder includes an earloop extending from a proximal end to a distal end. The magnetic radiographic marker holder includes a marker holder body coupled to the earloop at the distal end of the earloop. The magnetic radiographic marker holder includes a magnet coupled to the marker holder body to receive and magnetically couple a radiographic marker to the marker holder body.

According to other aspects of the present disclosure, the magnetic radiographic marker holder may include one or more of the following features. The marker holder body may include a recess formed in a surface of the marker holder body and the magnet may be arranged within the recess to couple the magnet to the marker holder body. The marker holder body may include a cavity formed within the marker holder body and the magnet may be arranged within the cavity to couple the magnet to the marker holder body. The earloop may be composed of a flexible material. The magnetic radiographic marker holder may further include an internal wire arranged within the earloop, wherein the internal wire is composed of a flexible material. The flexible material may be a rubber. The rubber may be at least one of a medical-grade silicon rubber or a nylon rubber. The flexible material may be an elastomer. The elastomer may be at least one of a thermoplastic elastomer or a polyurethane elastomer. The flexible material may be a three-dimensional (3D) printable material. The 3D printable material may include one of a silicon material or a resin material. The earloop and marker holder body may be composed of radiolucent materials. The earloop may be sized such that when worn on an ear of a patient the marker holder body is arranged adjacent a particular spinal region of the patient. The particular spinal region may include a cervical spinal region containing at least one of a C1 spinal level or a C2 spinal level. The magnetic radiographic marker holder may further include the radiographic marker magnetically coupled to the marker holder body, the radiographic marker including a marker body, a patient identification code on the marker body, and a magnet for magnetic attachment of the radiographic marker to the marker holder body. The patient identification code may be composed of a radiopaque material. The patient identification code may include at least one of a barcode or a QR code. The patient identification code may encode metadata information. The metadata information may include a consistency descriptor for a modified barium swallow study. The magnetic radiographic marker holder may further include a patient identification marker magnetically coupled to the marker holder body, the patient identification marker including a marker body, a patient identification code on the marker body, and a magnet for magnetic attachment to the marker holder body. The patient identification code may include at least one of a barcode or a QR code. The marker body may include at least one of a contrasting color or a reflective surface configured to provide visibility in video or photographic imaging. The patient identification code may encode metadata information. The metadata information may include a consistency descriptor for a modified barium swallow study.

According to another aspect of the present disclosure, a magnetic radiographic marker holder is provided. The magnetic radiographic marker holder includes a first earloop extending from a proximal end to a distal end. The magnetic radiographic marker holder includes a first marker holder body coupled to the first earloop at the distal end of the first earloop. The magnetic radiographic marker holder includes a first magnet coupled to the first marker holder body to receive and magnetically couple a radiographic marker to the first marker holder body. The magnetic radiographic marker holder includes a second earloop extending from a proximal end to a distal end. The magnetic radiographic marker holder includes a second marker holder body coupled to the second earloop at the distal end of the second earloop. The magnetic radiographic marker holder includes a second magnet coupled to the second marker holder body to receive and magnetically couple a radiographic marker to the second marker holder body. The magnetic radiographic marker holder includes a neck band coupling the distal end of the first earloop to the distal end of the second earloop.

According to other aspects of the present disclosure, the magnetic radiographic marker holder may include one or more of the following features. The first marker holder body may have a first recess formed in a surface thereof and the first magnet may be arranged within the first recess to couple the first magnet to the first marker holder body. The second marker holder body may have a second recess formed in a surface thereof and the second magnet may be arranged within the second recess to couple the second magnet to the second marker holder body. The first marker holder body may have a first cavity formed therein and the first magnet may be arranged within the first cavity to couple the first magnet to the first marker holder body. The second marker holder body may have a second cavity formed therein and the second magnet may be arranged within the second cavity to couple the second magnet to the second marker holder body. The first earloop and the second earloop may be composed of a flexible material. The neck band may be composed of a flexible material. The neck band may be composed of a corded material.

According to another aspect of the present disclosure, a magnetic radiographic marker holder is provided. The magnetic radiographic marker holder includes a hairband extending from a first end to a second end. The magnetic radiographic marker holder includes a marker holder body coupled to the hairband at the first end of the hairband. The magnetic radiographic marker holder includes a magnet coupled to the marker holder body to receive and magnetically couple a radiographic marker to the marker holder body.

According to other aspects of the present disclosure, the magnetic radiographic marker holder may include one or more of the following features. The magnetic radiographic marker holder may further include a second marker holder body coupled to the hairband at the second end of the hairband, the second marker holder body having coupled thereto a second magnet to receive and magnetically couple a radiographic marker thereto. The marker holder body may have a recess formed in a surface thereof and the magnet may be arranged within the recess to couple the magnet to the marker holder body. The marker holder body may have a cavity formed therein and the magnet may be arranged within the cavity to couple the magnet to the marker holder body. The magnetic radiographic marker holder may further include at least one decorative embellishment coupled to the hairband. The at least one decorative embellishment may include two animal ears. The at least one decorative embellishment may include a unicorn horn.

According to another aspect of the present disclosure, a magnetic radiographic marker holder is provided. The magnetic radiographic marker holder includes a headband forming a loop to be worn around a circumference of a patient's head. The magnetic radiographic marker holder includes a marker holder body coupled to the headband. The magnetic radiographic marker holder includes a magnet coupled to the marker holder body to receive and magnetically couple a radiographic marker to the marker holder body.

According to other aspects of the present disclosure, the magnetic radiographic marker holder may include one or more of the following features. The marker holder body may have a recess formed in a surface thereof and the magnet may be arranged within the recess to couple the magnet to the marker holder body. The marker holder body may have a cavity formed therein and the magnet may be arranged within the cavity to couple the magnet to the marker holder body.

### BRIEF DESCRIPTION OF THE DRAWINGS

FIG. 1 illustrates an example magnetic marker holder having an earloop construction in accordance with some examples described in the present disclosure.

FIG. 2 illustrates an example magnetic marker holder having an alternative earloop construction in accordance with some examples described in the present disclosure.

FIG. 3 illustrates an example magnetic marker holder having an earloop construction being worn by a patient.

FIG. 4 illustrates an example magnetic marker holder having a dual earloop construction being worn by a patient.

FIG. 5 illustrates an example magnetic marker holder having a hairband construction being worn by a patient.

FIG. 6 illustrates an example magnetic marker holder having a headband construction being worn by a patient.

FIG. 7 illustrates an example magnetic marker holder having an earloop construction and a patient identification marker.

### DETAILED DESCRIPTION

Described here are magnetic radiographic marker holders for use with x-ray imaging and other imaging modalities. Advantageously, the magnetic radiographic marker holders described in the present disclosure enable repeatable positioning of radiographic markers relative to the patient’s anatomy. The magnetic radiographic marker holders enable swapping of different radiographic markers without changing the relative positioning between the attached radiographic marker(s) and the patient anatomy. In some implementations, the magnetic radiographic marker holders may also be used with patient identification markers for imaging studies beyond x-ray imaging, including but not limited to video swallow studies, endoscopic imaging, or photographic documentation.

In general, the magnetic radiographic marker holders include a marker holder body to which a radiographic marker can be magnetically coupled. For instance, the marker holder body can have a magnet coupled thereto (e.g., attached to, arranged within, etc.) and the radiographic marker can be magnetically coupled to the marker holder body via the magnet. In some examples, the radiographic marker can have its own magnet that can be magnetically coupled to the magnet of the marker holder body. This construction enables consistent, repeatable positioning of radiographic markers on the marker holder body. The magnetic radiographic marker holder itself can include a number of different configurations, including an earpiece worn on a patient’s ear, a hairband, a headband, or the like.

Advantageously, the magnetic radiographic marker holders described in the present disclosure attach directly to a patient and can move with the patient. Additionally, by their design and construction the magnetic radiographic marker holders allow for consistent and repeatable placement of radiographic markers. Together, these features enable tighter collimation of the radiation beam, which allows for reducing the radiation exposure to the patient. As a non-limiting example, in swallowing studies the magnetic radiographic marker holders enable consistent placement of radiographic markers adjacent the cervical spine region of the patient, which allows for the radiographic markers to be seen in x-ray images without covering anatomical regions important to the imaging study. In some implementations, the magnetic radiographic marker holders may also help ensure correct patient identification during imaging studies by receiving patient identification markers with encoded patient information. Additionally, the magnetic radiographic marker holders may enable automated metadata entry through encoded markers, such as barcodes or QR codes, that can be scanned by post-processing software.

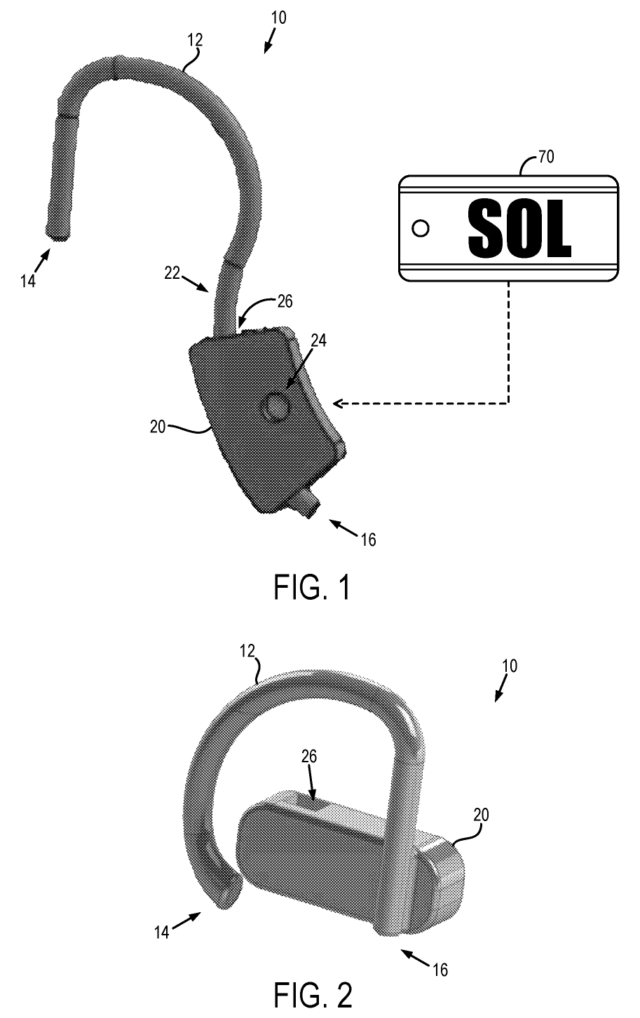

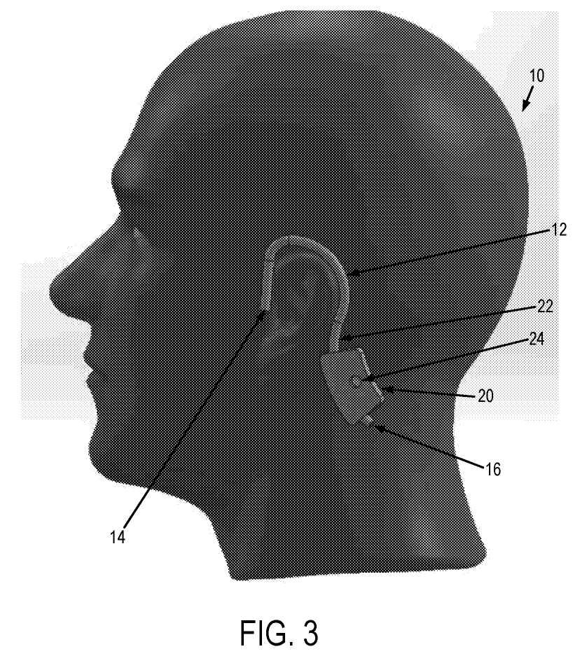

FIGS. 1–3 illustrate example magnetic marker holder 10 according to some embodiments described in the present disclosure. The magnetic marker holder 10 generally includes an earloop 12 extending from a proximal end 14 to a distal end 16 and a marker holder body 20 coupled to the earloop 12 at its distal end 16. These components work together to provide a secure and adjustable device for attaching radiopaque or other radiographic markers during x-ray imaging studies, including modified barium swallow studies.

The earloop 12 is dimensioned such that the distal end 16 extends below the patient’s ear to position the marker holder body 20 over or otherwise adjacent a particular spinal level of the patient. As a non-limiting example, the spinal level may be in a cervical spinal region of the patient, such as the C1 spinal level, the C2 spinal level, between the C1 and C2 spinal level, and so on. The positioning of the marker holder body 20 over the C1/C2 area of the spine may be advantageous for imaging purposes. This location may allow for optimal visibility of the radiographic marker 70 in x-ray images while minimizing interference with the structures being observed during the imaging study. In this way, a radiographic marker 70 that is magnetically coupled to the marker holder body 20 will be arranged relative to the patient’s anatomy at a location that keeps the radiographic marker 70 from obscuring anatomy relevant to the imaging study while also allowing for tighter, more efficient collimation of the x-ray beam, which enables reducing the radiation beam exposure to the patient.

The earloop 12 may be designed to securely attach to a patient’s ear during an x-ray imaging study, including a modified barium swallow study. In some examples, the earloop 12 may have a curved shape that conforms to the contours of the outer ear. The earloop 12 may be designed with a smooth surface and rounded edges to minimize irritation and ensure patient comfort during the procedure. In some examples, the earloop 12 may include a soft, cushioned pad at the point of contact with the ear to further enhance comfort and prevent slippage.

The earloop 12 may be constructed from a variety of flexible, biocompatible materials that ensure comfort and a secure fit on the patient’s ear. In some examples, the earloop 12 is may be composed of a material that is resistant to radiation, such that the earloop 12 does not degrade when exposed to x-ray radiation; is radiolucent, such that the earloop 12 is not visible in x-ray images; can be sterilized easily, such that the magnetic marker holder 10 may be used multiple times by sterilizing the earloop 12 between uses; and is soft or otherwise flexible, such that the earloop 12 can be comfortably worn by the patient. In some examples, the earloop 12 may be made from medical-grade silicone rubber, which is radiation resistant, soft, radiolucent, and can be sterilized easily. Alternatively, the earloop 12 may be composed of a thermoplastic elastomer (TPE), thermoplastic polyurethane (TPU), a polyurethane elastomer, nylon rubber, additive manufacturing build materials (e.g., resins, plastics, etc.), or the like.

To enhance the stability and adjustability of the magnetic marker holder 10, an internal metal wire (not shown) may be incorporated into the earloop 12. As a non-limiting example, the wire may be made from titanium, which offers good strength-to-weight ratio and corrosion resistance. Alternatively, the wire may be composed of beryllium copper wire, stainless steel, phosphor bronze, or other materials with suitably strength and flexibility properties that provide durability and fatigue resistance.

The dimensions of the earloop 12 may vary to accommodate different patient sizes and anatomies. In some examples, the earloop 12 may have a length ranging from 100 to 150 millimeters and a thickness of 2 to 5 millimeters. These dimensions may be adjusted based on specific patient requirements or clinical preferences. In some implementations, the earloop 12 may be available in multiple sizes to accommodate different ear anatomies. For example, the magnetic marker holder 10 may include small, medium, and large earloop 12 size options. Alternatively, the earloop 12 may be adjustable, such as by including a bendable internal wire core, as described above, that can be manipulated to provide a customized fit of the earloop 12 for individual patients.

For patients with unique anatomical considerations, such as those without an ear, alternative attachment methods for the magnetic marker holder 10 may be provided. As one non-limiting example, the magnetic marker holder 10 may include a tapeable magnet with adhesive properties similar to a bandage. This magnet may be affixed under the patient’s external auditory canal (EAC) to provide an attachment point for the magnetic marker holder 10 when a traditional earloop 12 cannot be used. In still other examples, the marker holder body 20 may be coupled to a hairband, a headband, or other such means for coupling the marker holder body 20 to the patient. Some examples of alternative configurations are provided in more detail below.

In some examples, the distal end 16 of the earloop 12 may include an extended portion 22 to which the marker holder body 20 can be securely coupled. The extended portion 22 may be flexible, allowing for adjustment to fit the contours of individual patients. This flexibility may help ensure proper positioning of the marker holder 20 while maintaining patient comfort. The material used for the extended portion 22 may be selected to provide an appropriate balance of rigidity for stability and flexibility for customization. In some instances, the extended portion 22 may be composed of the same material as the earloop 12, while in other instances the extended portion 22 may be composed of a different material.

The extended portion 22 may be adjustable in length. In some implementations, the extended portion 22 may be composed of modular segments that can be added or removed to adjust the overall length of the earloop 12. In some examples, the extended portion 22 may be rotatable at its connection point with the earloop 12. This feature may allow for fine-tuning of the marker holder body 20 position without the need to adjust the earloop 12 placement. The rotation mechanism may include detents or other features to maintain the chosen position during use.

The extended portion 22 may also incorporate a curvature or bend to better follow the natural contours of the patient’s head and neck. This curvature may be preformed in some implementations, while in others, the extended portion 22 may be flexible (e.g., via an internal wire as described above), allowing a healthcare provider to customize the shape of the earloop 12 for each patient.

In some implementations, the extended portion 22 may include markings or measurements along its length. These markings may assist healthcare providers in consistently positioning the marker holder body 20 across different patients or procedures.

The marker holder body 20 is designed to securely hold radiographic markers of various shapes and sizes. The marker holder body 20 can be sized and shaped to accommodate various sizes and shapes of radiographic markers 50. This design flexibility may allow clinicians to use different radiographic marker types depending on the specific requirements of the study or patient. The marker holder body 20 may be constructed from various rigid materials to provide structural integrity. Like the earloop 12, the marker holder body 20 is preferably composed of a material that is resistant to radiation, such that the marker holder body 20 does not degrade when exposed to x-ray radiation; is radiolucent, such that the marker holder body 20 is not visible in x-ray images; and can be sterilized easily. As one non-limiting example, the marker holder body 20 may be composed of a thermoplastic, such as polyether ether ketone (PEEK), which has favorable strength and biocompatibility properties. Alternatively, the marker holder body 20 may be composed of other plastic or polymer materials, such as acrylonitrile butadiene styrene (ABS), polycarbonate, or suitable build materials for additive manufacturing, such as polylactic acid (PLA), resins, or the like.

In some configurations, the marker holder body 20 can include a channel 26 through which the earloop 12 is passed to couple the marker holder body 20 to the ear loop 12. In some other configurations, the marker holder body 20 may be coupled to the earloop 12 through other means, such as by clips. In still other configurations, the marker holder body 20 may be integrally formed with the ear loop 12.

In general, the marker holder body 20 allows for rapid changing of radiographic markers 50 during an imaging study. For example, in an MBS study, a clinician may quickly remove one radiographic marker 70 and replace it with another as different consistencies of barium contrast are administered to the patient. This quick-change capability may help improve the efficiency of the study and reduce overall procedure time.

As one example, the marker holder body 20 may utilize a magnetic attachment, such as by using a magnet 24 embedded in or otherwise coupled to the marker holder body 20. The marker holder body 20 may have multiple attachment points for the radiographic marker 70. For example, the marker holder body 20 may include more than one magnet 24, such as one magnet on each side of the marker holder body 20. This configuration may allow the clinician to choose which side to attach the radiographic marker 70 based on patient positioning or other factors. When the marker holder body 20 implements magnetic attachment of the radiographic marker 70, the magnet 24 may be coupled to the marker holder body 20. In some configurations, a recess (not shown) is formed in a surface of the marker holder body 20 and the magnet 24 may be arranged within the recess. In some other configurations, a cavity 26 may be formed in the marker holder body 20 and the magnet 24 may be placed or otherwise arranged within the cavity 26 of the marker holder body 20. The magnet 24 may be strong enough to securely hold a radiographic marker 70 in place during an imaging study while allowing for easy removal when changing radiographic markers.

In some examples, the marker holder body 20 may incorporate non-magnetic options for coupling the radiographic marker 70 to the marker holder body 20. For example, mechanical fasteners such as clips, snap-fit connections, or hook-and-loop fasteners, or the like, may be employed for attaching or otherwise removably coupling radiographic markers 50 to the marker holder body 20. In these cases, the corresponding fasteners will be arranged on the marker holder body 20 and radiographic marker 70. These alternative attachment mechanisms may provide options for facilities that prefer non-magnetic solutions or in cases where magnetic interference is a concern.

Compatible radiographic markers 50 for use with the magnetic marker holder 10 may include various types. In some examples, the radiographic markers 50 may be thin metal plates or discs with radiopaque markings or symbols formed thereon or otherwise coupled thereto. These radiographic markers 50 may have a ferromagnetic backing or component that allows for magnetic attachment to the marker holder body 20. For example, in some cases the radiographic marker 70 may have a magnet coupled thereto for magnetic attachment to the magnet 24 of the marker holder body 20. In still other implementations, the radiographic markers 50 themselves may be magnetic and adhere to a metal surface on the marker holder body 20. In such instances, the marker holder body 20 may not have a magnet 24, and instead the magnetic attachment of the radiographic marker 70 may be provided by the radiographic marker 70 itself being magnetic.

In some implementations, the magnetic marker holder 10 may be provided as part of a kit that includes specially designed radiographic markers 70. In some cases, commercially available radiographic markers may not be functional with the magnetic marker holder 10, and thus specially designed radiographic markers 70 may be provided. The specially designed radiographic markers 70 may have a size configured for rapid handling, such that a clinician can quickly grasp and manipulate the radiographic marker 70 during an imaging study. The specially designed radiographic markers 70 may also have markings or numbers that are visible in low-light conditions, such as in a dark room used during fluoroscopy procedures. Additionally, the specially designed radiographic markers 70 may have a weight configured to not interfere with the functioning of the magnetic marker holder 10 during an examination. In some examples, the specially designed radiographic markers 70 may include an integrated magnet arranged within or coupled to the radiographic marker 70 for magnetic attachment to the magnet 24 of the marker holder body 20. The combination of these features may enable efficient and reliable use of the radiographic markers 70 with the magnetic marker holder 10 during imaging studies.

As described above, in some embodiments the magnetic marker holder 10 is configured to include a single earloop 12 for attachment to a patient’s ear. In alternative configurations, the magnetic marker holder 10 may include two earloops 12 or the earloop 12 may be replaced with other attachment mechanisms, such as a hairband, a headband, or the like.

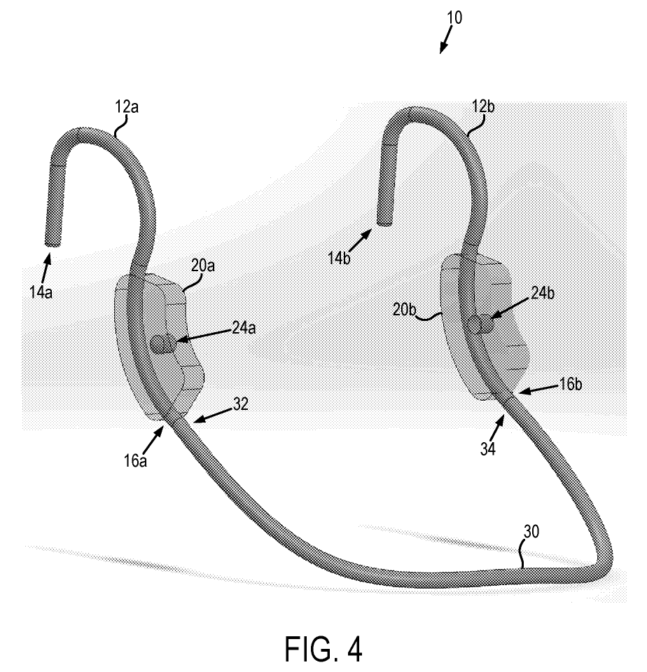

Referring to FIG. 4, in another example of the magnetic marker holder 10, the marker holder body 20 may be coupled to a pair of earloops 12 that are coupled together by a neck band 30. In these instances, the magnetic marker holder device 10 may include a first earloop 12aextending from a proximal end 14a to a distal end 16a and a second earloop 12b extending from a proximal end 14b to a distal end 16b. The first earloop 12ais coupled to the second earloop 12b by a neck band 30 that couples on its first end 32 to the distal end 16a of the first earloop 12a and on its second end 34 to the distal end 16b of the second earloop 12b. The magnetic marker holder device 10 may include a single marker holder body 20 (e.g., coupled to the distal end 16aof the first earloop 12a or the distal end 16b of the second earloop 12b), or may include a pair of marker holder bodies: a first marker holder body 20a coupled to the distal end 16a of the first earloop 12a and a second marker holder body 20b coupled to the distal end 16b of the second earloop 12b. In these cases, the first marker holder body 20a may have a first magnet 24aand the second marker holder body 20b may have a second magnet 24b.

The neck band 30 may be composed of a flexible material. In some implementations, the neck band 30 may be composed of the same material as the earloops 12. Alternatively, the neck band 30 may be composed of a different material than the earloops 12. As one example, the neck band 30 may be composed of a string or other corded material.

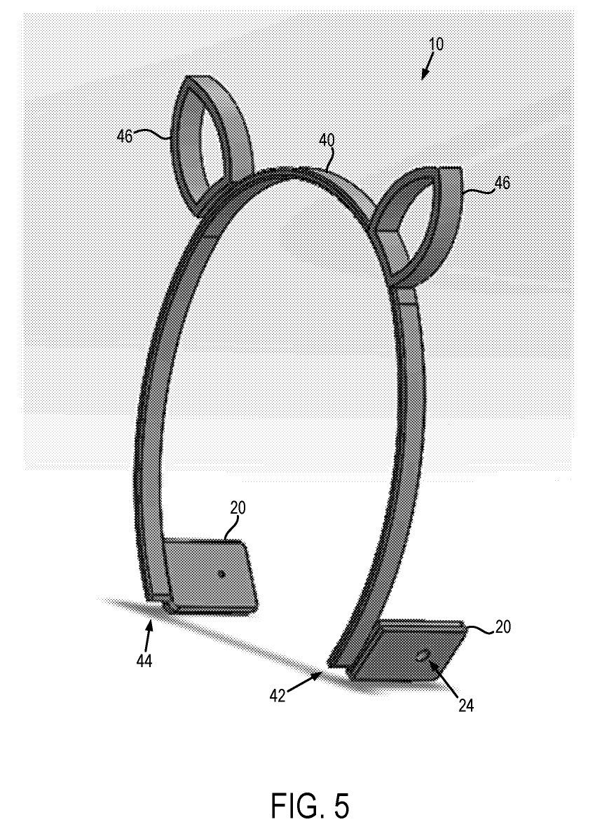

As illustrated in FIG. 5, in one example, the marker holder body 20 may be coupled to a hairband 40. This design is advantageous for pediatric patients. In these cases, the magnetic marker holder 10 may be designed to accommodate smaller patient sizes. This hairband-style configuration may allow for secure placement on pediatric patients who may have smaller or differently shaped ears compared to adult patients. The hairband-style design may wrap around the patient’s head, providing stability and comfort during the swallow study procedure. In some examples, the marker holder body 20 coupled to the hairband 40 may be positioned to align with the patient’s spine, such as in the area of the C1 and C2 vertebrae. This positioning may allow for optimal placement of the radiographic marker 70 within the desired viewing window during the imaging study. The marker holder body 20 may also be designed to be flexible or adjustable to accommodate different neck lengths and spine curvatures in pediatric patients. This adjustability may allow for proper positioning of the radiographic marker 70 regardless of the patient’s specific anatomy.

The hairband 40 extends from a first end 42 to a second end 44. The marker holder body 20 may be coupled to the hairband at the first end 42 or at the second end 44. Additionally or alternatively, the magnetic marker holder device 10 may include a pair of marker holder bodies: one marker holder body 20 coupled to the first end 42 of the hairband 40 and a second marker holder body 20 coupled to the second end of the hairband 40. The hairband 40 may be adjustable to accommodate a range of head sizes within the pediatric patient population. For instance, the hairband 40 may incorporate adjustable fasteners or elastic materials to ensure a secure and comfortable fit for various pediatric head sizes.

To increase patient compliance and comfort, especially for younger pediatric patients, the pediatric version of the magnetic marker holder 10 may incorporate child-friendly features. For example, the hairband 40 may be embellished with decorative elements 46 that are appealing to pediatric patients. By way of example, the hairband 40 can be embellished with decorative elements 46 such as animal ears (e.g., cat ears), a unicorn horn, or other such whimsical elements. These playful additions may help distract and engage pediatric patients, potentially reducing anxiety and improving cooperation during imaging studies.

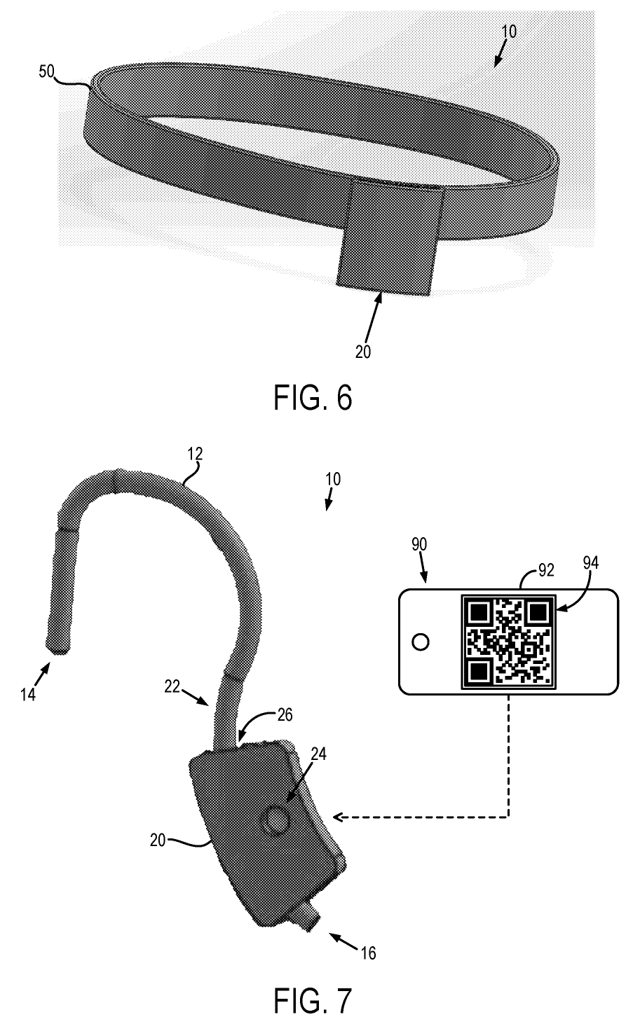

As illustrated in FIG. 6, in another example, the marker holder body 20 may be coupled to a stretchable or otherwise flexible headband 50. The headband 50 may be composed of any suitable material, such as elastic materials, stretchable fabrics, or the like. The marker holder body 20 may be adjustable along the length of the headband 50, or may be in a fixed position. In some implementations, an extension arm (not shown) may couple the marker holder body 20 to the headband 50 to allow for positioning of the marker holder body 20 to align with the patient’s spine, such as in the area of the C1 and C2 vertebrae. This wraparound version of the magnetic marker holder 10 may provide increased stability, which may be beneficial for patients who tend to move frequently during an imaging procedure. The wraparound design may help ensure the radiographic marker 70 remains properly positioned even with patient movement.

As one example use, the magnetic marker holders 10 described in the present disclosure may be used during a modified barium swallow (MBS) study to enhance efficiency, reduce radiation exposure, and improve image quality. In some cases, the device may be attached to a patient's ear prior to beginning the study.

During the MBS study, a clinician may easily attach and detach different radiographic markers 70 to the marker holder body 20 as needed for various consistencies of barium contrast. The consistent positioning of the radiographic marker 70 may allow for more precise collimation of the x-ray beam, potentially reducing overall radiation exposure to the patient and staff.

Advantageously, the magnetic marker holder 10 may remain securely in place even if the patient moves, ensuring that the radiographic marker 70 stays within the imaging field-of-view throughout the procedure. This consistent radiographic marker 50 placement may reduce the need for the radiologist to search for the marker location, potentially allowing more focus on observing swallowing mechanics and pathology.

The magnetic marker holder 10 may enable faster transitions between different consistencies during the study. A clinician may quickly swap radiographic markers 70 without needing to reposition markers or adjust collimation repeatedly. This efficiency may allow more swallows to be observed within a given time frame.

By potentially reducing procedure time and enhancing image consistency, the magnetic marker holder 10 may allow for more thorough assessment of swallowing function while minimizing radiation exposure. The improved efficiency may also enable clinicians to perform more studies within their allotted time, potentially increasing patient throughput.

Referring to FIG. 7, in some implementations, the magnetic marker holder 10 may be used with a patient identification marker 90. In some medical imaging studies, a radiographic marker 70 may not be highly visible or may not be suitable for the imaging modality being used. For example, in video swallow studies, photographs, endoscopic imaging, or other non-x-ray imaging modalities, radiopaque markers may not provide adequate visibility. In these instances, the patient identification marker 90 may be configured to be visible on the particular imaging modality. For example, the patient identification marker 90 may be optically visible in video frames, photographs, or other imaging formats. The patient identification marker 90 may include a marker body 92, a patient identification code 94 on the marker body 92, and a magnet 96 for magnetic attachment to the marker holder body 20. The marker body 92 may be composed of materials or include surface features that provide visibility in the relevant imaging modality, such as contrasting colors, reflective surfaces, or other optically detectable characteristics for video or photographic imaging. The patient identification code 94 may be a barcode, a QR code, or other machine-readable code that encodes patient identification information. The patient identification marker 90 may be magnetically attached to the marker holder body 20 of the magnetic marker holder 10 to hold patient identifiers in imaging studies such as an esophagram, upper GI study, video swallow studies, or other imaging of the head and neck. This configuration may help ensure that images are associated with the correct medical record number (MRN) after image acquisition, which may reduce errors in busy radiology practices where images may otherwise be associated with incorrect patients during post-processing.

In some implementations, the radiographic markers 70 and/or patient identification marker 90 described herein may additionally or alternatively encode metadata information. For example, the patient identification code 94 on the marker body 92 may encode metadata information, such as consistency descriptors (e.g., thin, mildly thick, moderately thick, or pudding) used in modified barium swallow studies. Post-processing software may scan the barcode or QR code on images and automatically fill in the corresponding metadata on toggleable metadata fields in existing radiology images. This automated metadata entry may be used instead of or in combination with radio-opaque numbers on the radiographic markers 70. In some examples, the radiographic markers 70 may include a radiopaque code, such as a radiopaque barcode or radiopaque QR code, that is visible in x-ray images and encodes metadata information for the imaging study. The radiopaque code may be formed from radiopaque materials or markings on the radiographic marker 70, such that the code appears in x-ray images and can be scanned by post-processing software to automatically populate metadata fields. This configuration may allow for automated metadata entry directly from x-ray images without requiring a separate optically visible marker. In some examples, the patient identification marker 90 may include both patient identification information and metadata information encoded in one or more barcodes or QR codes. The post-processing software may scan the codes and verify that the correct images are associated with the correct patient while also automatically populating metadata fields.

The present disclosure has described one or more preferred embodiments, and it should be appreciated that many equivalents, alternatives, variations, and modifications, aside from those expressly stated, are possible and within the scope of the invention.

1. A magnetic radiographic marker holder, comprising:
an earloop extending from a proximal end to a distal end;
a marker holder body coupled to the earloop at the distal end of the earloop; and
a magnet coupled to the marker holder body to receive and magnetically couple a radiographic marker to the marker holder body.

2. The magnetic radiographic marker holder of claim 1, wherein the marker holder body comprises a recess formed in a surface of the marker holder body and the magnet is arranged within the recess to couple the magnet to the marker holder body.

3. The magnetic radiographic marker holder of claim 1, wherein the marker holder body comprises a cavity formed within the marker holder body and the magnet is arranged within the cavity to couple the magnet to the marker holder body.

4. The magnetic radiographic marker holder of claim 1, wherein the earloop is composed of a flexible material.

5. The magnetic radiographic marker holder of claim 4, further comprising an internal wire arranged within the earloop, wherein the internal wire is composed of a flexible material.

6. The magnetic radiographic marker holder of claim 4, wherein the flexible material is one of a rubber, an elastomer, or a three-dimensional (3D) printable material.

7. The magnetic radiographic marker holder of claim 1, wherein the earloop and marker holder body are composed of radiolucent materials.

8. The magnetic radiographic marker holder of claim 1, wherein the earloop is sized such that when worn on an ear of a patient the marker holder body is arranged adjacent a particular spinal region of the patient.

9. The magnetic radiographic marker holder of claim 8, wherein the particular spinal region comprises a cervical spinal region containing at least one of a C1 spinal level or a C2 spinal level.

10. A magnetic radiographic marker holder, comprising:
a first earloop extending from a proximal end to a distal end;
a first marker holder body coupled to the first earloop at the distal end of the first earloop;
a first magnet coupled to the first marker holder body to receive and magnetically couple a radiographic marker to the first marker holder body;
a second earloop extending from a proximal end to a distal end;
a second marker holder body coupled to the second earloop at the distal end of the second earloop;
a second magnet coupled to the second marker holder body to receive and magnetically couple a radiographic marker to the second marker holder body; and
a neck band coupling the distal end of the first earloop to the distal end of the second earloop.

11. The magnetic radiographic marker holder of claim 10, wherein the first marker holder body has a first recess formed in a surface thereof and the first magnet is arranged within the first recess to couple the first magnet to the first marker holder body.

12. The magnetic radiographic marker holder of claim 10, wherein the second marker holder body has a second recess formed in a surface thereof and the second magnet is arranged within the second recess to couple the second magnet to the second marker holder body.

13. The magnetic radiographic marker holder of claim 10, wherein the first marker holder body has a first cavity formed therein and the first magnet is arranged within the first cavity to couple the first magnet to the first marker holder body.

14. The magnetic radiographic marker holder of claim 10, wherein the second marker holder body has a second cavity formed therein and the second magnet is arranged within the second cavity to couple the second magnet to the second marker holder body.

15. A magnetic radiographic marker holder, comprising:
a hairband extending from a first end to a second end;
a marker holder body coupled to the hairband at the first end of the hairband; and
a magnet coupled to the marker holder body to receive and magnetically couple a radiographic marker to the marker holder body.

16. The magnetic radiographic marker holder of claim 15, further comprising a second marker holder body coupled to the hairband at the second end of the hairband, the second marker holder body having coupled thereto a second magnet to receive and magnetically couple a radiographic marker thereto.

17. The magnetic radiographic marker holder of claim 15, wherein the marker holder body has one of a recess formed in a surface thereof or a cavity formed therein, and the magnet is arranged within the recess or the cavity to couple the magnet to the marker holder body.

18. The magnetic radiographic marker holder of claim 15, further comprising at least one decorative embellishment coupled to the hairband.

19. A magnetic radiographic marker holder, comprising:
a headband forming a loop to be worn around a circumference of a patient’s head;
a marker holder body coupled to the headband; and
a magnet coupled to the marker holder body to receive and magnetically couple a radiographic marker to the marker holder body.

20. The magnetic radiographic marker holder of claim 19, wherein the marker holder body has one of a recess formed in a surface thereof or a cavity formed therein, and the magnet is arranged within the recess or the cavity to couple the magnet to the marker holder body.
